(19) United States
(12) Patent Application Publication
(10) Pub. No.: US 2004/0082062 A1
(43) Pub. Date: Apr. 29, 2004

(54) Method and medium for in vitro culture of human embryos

(76) Inventors: Sarah Robertson, St. Peters (AU); Matts F. Wikland, Goteborg (SE); Cecilia Sjoblom, Goteborg (SE)

Correspondence Address: Coleman, Sudol, Sapone, P.C., 714 Colorado Ave., Bridgeport, CT, 06605-1601, US

(21) Appl. No.: 10/631,395

(22) Filed: Jul. 31, 2003

Related U.S. Application Data
(63) Continuation-in-part of application No. 09/720,231, filed on Feb. 20, 2001, now Pat. No. 6,605,468.
Application No. 09/720,231 is a 371 of international application No. PCT/AU99/00499, filed on Jun. 18, 1999.

(30) Foreign Application Priority Data
Jun. 19, 1998 (AU) PP4212

(51) Int. Cl. C12N 5/08; C12N 5/02

(52) U.S. Cl. 435/366; 435/404

(57) ABSTRACT

Disclosed is a medium for the propagation of early stage embryos to blastocyst stage. The medium contains an effective amount of human GM-CSF to increase the percentage of pre-blastocyst embryos which develop to transfer ready blastocysts. Also disclosed is a method of growing early stage human embryos to transfer ready blastocysts. The method includes the step of incubating the embryos in vitro in a culture medium containing an effective amount of human GM-CSF for a time and under conditions to increase the proportion of transfer ready blastocysts. An IVF program that includes the method of growing early stage human embryos to transfer ready blastocysts is also disclosed.

### RELATED APPLICATIONS

This application is a continuation-in-part application of Ser. No. 09/720,231, filed Feb. 20, 2001, which is a §371 of PCT/AU99/00499, filed Jun. 18, 1999.

### BACKGROUND OF THE INVENTION

Infertility is a great concern to many couples who wish to conceive. The proportion of couples that are unable to conceive naturally is remarkably high. In the USA it is said that some 10-15% of couples of reproductive age are unable to have children, w hereas in the United Kingdom the proportion has been estimated at 14%.

In the last 20 years or so some hope has been held out to infertile couples with the development of in vitro fertilisation (IVF) techniques. These IVF techniques generally take the form of stimulating the female to ovulate, contacting collected ova with sperm in vitro and introducing fertilised ova into the uterus. Multiple variations of this general process also exist. Despite considerable research and technical advances in the IVF field the rate of successful pregnancy following IVF treatment is still quite low and is in the order of 15 to 25% per cycle.

Undertaking an IVF program often causes great anguish, especially where there is no resultant successful pregnancy. It is presently believed that the poor success rate for IVF treatment is due to an extraordinarily high rate of early embryonic loss or implantation failure (Weinberg et al., 1988; Lenton et al., 1988).

The low efficacy of IVF, together with its high cost and the associated psychological trauma from repeated treatment failures make it desirable that improvements are made to the procedure. Current methods of increasing pregnancy rates during IVF treatment include placing multiple embryos (2-5) into the uterine cavity. This is not always successful, and also carries with it a higher risk of multiple pregnancy.

In most in vitro fertilisation units embryos are transferred to the uterus 2 days after fertilisation (4-8 cells). One view is that the use of embryos at this early stage may contribute significantly to the low pregnancy outcome of IVF programs, and that it is more desirable to use embryos at the blastocyst stage reached at day 5-7 of culture. The advantages suggested include improved synchronisation between embryo and uterus and the ability to select better quality embryos over the longer culture period. Blastocyst transfer may also help reduce the number of multiple births resulting from IVF, through allowing the selection of fewer numbers of highly competent embryos per transfer.

Unfortunately in standard culture media the majority of embryos (about 75%) fail to develop beyond the 4-8 cell stage. Nevertheless with certain clinical indications implantation of human embryos is performed at the blastocyst stage despite the low proportions of embryos that develop to blastocyst.

Media used in early efforts were simply media used for general tissue culture methods, which were loosely formulated to reflect concentrations of a range of components found in blood plasma. Embryo media generally in current use is somewhat different in that components loosely reflect components found in the female reproductive tract. Furthermore it is general practice that the embryo specific media is changed sequentially, typically with a first phase formulation used for pre compacted embryos (8 cells or less) and a second phase formulation used for compacted embryos. The first phase media is used to culture embryos to day 3 although at times this might be extended to day 4 or beyond, and in rare instances this might be used for culturing embryos up to day 2 stage. Thus the first phase media is generally used for embryos to the 8 cell stage (corresponding to day 3) or up to the morula stage. These embryos are referred herein as being pre-compacted embryos. Furthermore the second phase media is generally used for embryos once transferred out of first stage medium, and would generally be used from 8-cell stage or beyond. These embryos are referred to herein as post-compacted stage.

Some recent studies have used co-culture techniques whereby embryos are co-cultured with feeder cells, for example Vero cells, which technique can more than double blastocyst formation rates (Ménézo et al., 1990; Plachot et al., 1995). There have been a number of studies using these co-culture techniques which have shown increased implantation rates after blastocyst transfer (Ménézo et al., 1992), particularly in women with repeated previous implantation failures (Oliveness et al., 1955; Plachot et al., 1955). Co-culture is time consuming and expensive and concerns have been expressed about possible transfer of disease from contaminated cultures (Oliveness et al., 1955), in particular there is a concern relating to viral contamination which contamination is considered to be virtually impossible to fully eliminate. A safer and more practical approach is to attempt to produce a culture medium able to sustain embryo development through to the blastocyst stage that is independent of co-culture.

One approach to enhance in vitro embryo development without using co-culture techniques is to attempt to define factors that might be used to enhance embryo development in in vitro culture. A number of attempts have been already made to identify factors that might assist and amongst the promising factors are various stimulatory factors known as cytokines. One such factor, leukaemia inhibitory factor (LIF) has already been indicated as being positive in this regard for humans (Dunglison et al 1996) and livestock species, U.S. Pat. No. 5,418,159.

One of the many factors also currently under investigation in both animals and humans relative to conception and embryo development is granulocyte-macrophage colony-stimulating factor (GM-CSF). However to date there has been no definite indication that a medium supplemented with GM-CSF would be sufficient to enhance the in vitro development of embryos to the blastocyst stage in a defined culture medium.

GM-CSF is a 23-29 kD glycoprotein which although secreted in a soluble form in vitro, is one of many cytokines known to be sequestered and immobilised in the ECM (extracellular matrix) in vivo through association with heparan sulphate. GM-CSF was originally characterised as a hemopoietic regulator and determinant of the maturation and behaviour of myeloid leukocytes in peripheral tissues. It is now known that GM-CSF is produced by a diversity of cell types including T-lymphocytes, monocytes, macrophages, fibroblasts, endothelial cells and epithelial cells.

The uterine epithelium has been identified by in situ hybridisation and in in vitro cell isolation studies as a major source of GM-CSF in the mouse uterus (Robertson et al 1992, Robertson et al 1994) and human oviduct and uterus (Zhao and Chegini 1994, Giacomini et al 1995). A role for GM-CSF in reproductive processes was supported by studies perturbing the cytokine environment during early pregnancy in vivo (Tartakovsky and BenYair, 1991) and experiments showing impaired fertility in genetically GM-CSF deficient mice (Robertson et al 1999).

Studies of radio-labelled ligand binding show clearly that murine blastocysts bind ¹²⁵I-GM-CSF specifically, indicating that they express at least the low affinity form of the GM-CSF receptor. This conclusion was supported by RT-PCR analysis, which showed that blastocysts express mRNA for the α-subunit of the GM-CSF receptor complex (Robertson et al, 2001). A similar situation was found to exist in human embryos. GM-CSF-R mRNA was expressed at similar levels through the first four days of murine and human embryo development, from fertilisation to blastocyst stage. However mRNA for the β-subunit of the GM-CSF receptor complex was not detected in embryos of either species by the RT-PCR technique (Robertson et al, 2001, Sjoblom et al, 2002). Immunohistochemical experiments using specific antibodies with human embryos confirmed this result (Sjoblom et al, 2002). Together, these data suggest that embryos express GM-CSF receptor from at least as early as fertilisation, but that it may be of the low affinity form. The embryo therefore falls into the same category as endothelial cells and other non-hemopoietic cells which exhibit a biological response to GM-CSF despite expressing only low affinity receptors. Although it seems clear in hemopoietic cells that the α-subunit of the GM-CSF receptor cannot on its own transduce proliferative signal, it is not known whether the α-subunit can in some circumstances initiate responses in cells in the absence of the β-subunit. The recent discovery of unconventional forms of the GM-CSF receptor in the human suggests that this may be possible.

It has also been shown that binding of cognate ligands to the GM-CSF receptor α subunit in isolation may mediate increased glucose transport via a phosphorylation-independent pathway (Ding et al., 1994). Recent experiments by the inventor show that culture with recombinant mouse GM-CSF (mGM-CSF) stimulates increased glucose uptake in murine blastocysts, to an extent achievable with known glucose transport stimulants such as insulin-like growth factor-1, suggesting that this cytokine may stimulate metabolism in murine embryos.

There is some evidence to indicate that GM-CSF also participates in regulation of embryonic growth. Conditioned media rich in mGM-CSF have been found to be effective particularly in promoting blastocyst development in mice, particularly in the attachment of hatched blastocysts to serum attachment factors in plastic culture dishes (Robertson et al., 1991). The media was conditioned by cells from LPS activated mouse lung tissue, and contains a number of other factors which could contribute to the embryotrophic activity.

In further studies by the inventor one cell and eight cell mouse embryos were cultured with or without recombinant mouse GM-CSF (rm GM-CSF) in a defined medium, and again there was a modest improvement in blastocyst and post-blastocyst development The proportion of embryos developing to eight cell or blastocyst stage was not altered by cytokine. However the proportion of blastocysts hatched from the zona pellucida and attached to the culture dish was increased, and the speed of development accelerated, when GM-CSF was added to the culture dish (Robertson et al 1991; Robertson et al 2001).

In further experiments the survival and/or proliferation of blastomeres within developing mouse blastocysts, particularly inner mass cells, was shown to be enhanced by exposure to native GM-CSF in vivo, or by recombinant GM-CSF in vitro (Robertson et al., 1998; Robertson et al 2001).

Several groups have reported both positive and negative effects of GM-CSF on various stages of early embryo development. Hill et al. (1987) have found that GM-CSF at high doses (>1000 U/ml) inhibited the development of 2-cell embryos into morulae. In two studies, ectoplacental cone trophoblast has been found to proliferate in response to GM-CSF (Armstrong and Chaouat 1989; Lea and Clark 1993), but in the second instance an effect was obtained with native but not recombinant cytokine. Haimovoci et al. (1991) found that 250 U/ml or more of GM-CSF inhibited the attachment of blastocysts to fibronectin-coated culture dishes in the absence of serum. Lea and Clark (1993) have reported that recombinant GM-CSF (at between 10 and 100 U/ml) inhibited the incorporation of ³H-thymidine into outgrowing, implanted blastocysts, in a dose dependant manner. Tartakovsky and Ben-Yair (1991) found that systemic GM-CSF administration markedly enhanced early embryonic development in vivo, but did not note any effect of GM-CSF on embryonic development in vitro. These results are difficult to reconcile. However, the differences are likely to be related to the developmental stages examined, the methods for embryo culture, the strains of mice, and the sources and concentrations of cytokine used. For example, some cytokine preparations may contain potentially embryotoxic contaminants such as endotoxin. In addition, there is emerging evidence that there may be more than one mechanism by which GM-CSF is able to exert its effects in target cells, and it is possible that the glycosylation state of the cytokine (which would also be dependant upon its source) may be important for binding to unconventional receptors.

A study of bovine embryos (de Moraes and Hansen 1997) used recombinant bovine GM-CSF (rbGM-CSF) in attempt to enhance embryo development to blastocyst stage. The rbGM-CSF only had a significant impact on the proportion of embryos developing to blastocyst stage at very high levels of 10 ng/ml, and the numbers of embryos tested were relatively low so the results might be viewed with some concern. Additionally it was found however that the proportion of blastocysts that expanded or hatch dropped significantly with the 10 ng/ml rbGM-CSF and 1 ng/ml rbGM-CSF and thus can be seen an adverse effect on the capacity of the blastocysts to be used subsequently as their development had essentially terminated in vitro.

### SUMMARY OF THE INVENTION

The present invention results from a finding that recombinant human GM-CSF (rhGM-CSF) is effective at substantially increasing the proportion of early cleavage-stage embryos that develop to compacted morula and then blastocyst stage, and increasing the proportion of those embryos that continue to expanded blastocyst and then hatched blastocyst stages of development. The net result is that a much greater proportion of embryos can now be grown to blastocyst stage and used for implantation in an IVF program in humans. This contrasts with the mixed findings in other species, whereby only moderate and inconsistent effects on development to blastocyst stage and beyond were reported.

This finding has implications in the formulation of media for use in in vitro culturing of embryos to blastocyst stage and in methodologies of growing such embryos and in the manner in which IVF programs are conducted. It is anticipated that this invention will lead to a greater success rate in such IVF programs.

Thus in one broad form of a first aspect the invention could be said to reside in an embryo specific medium for propagation of early stage embryos to blastocyst stage, said medium containing an effective amount of human GM-CSF to increase the percentage of pre-blastocyst embryos which develop to transfer ready blastocysts.

Reference to ‘embryo specific’ when used in conjunction with ‘media’ means that the media have been formulated for, or are generally accepted as optimally suitable for, use in cultivating embryos, however it will be understood that they might also be useful for cultivation or maintenance of other cell types or tissues.

Transfer ready blastocysts are embryos developed to the stage where a blastoceol cavity is clearly evident and comprises greater than 50% of the volume of the embryo. This stage would in the in vivo situation normally be achieved 4-5 days after fertilisation, soon after the embryo has traversed the fallopian tube and arrives in the uterus.

In one form the medium is a serum deprived medium. The serum deprived medium is desirable in so far as the risk of contamination, typically by viruses, is drastically reduced. The term serum deprived when used in this specification refers to a medium that does not include serum, or any partially defined serum fraction as an additive, but may include a medium that includes serum derived components that have been substantially purified from serum, or recombinant serum components that have been prepared in the absense of serum, and may or may not have been modified.

In another form the medium might be a fully chemically defined medium, such media may include recombinant serum components that have been prepared in the absence of serum.

Most preferably the human GM-CSF is in purified form, and most preferably purified in from a non-animal and non-human source, and might thus be purified from a recombinant micro-organism.

The GM-CSF receptors of embryos appears to be somewhat unique in composition compared to GM-CSF receptors elsewhere and it is therefore likely that the support for embryo growth may not require a fully native GM-CSF. The hGM-CSF may thus be modified or altered in any one of a number of ways and may or may not need to be glycosylated. The hGM-CSF may be truncated, include amino acid deletions and substitutions or may be a recombinant molecule.

Where rhGM-CSF is used it is anticipated that the level of rhGM-CSF in the medium as used will be approximately 1 ng/ml which is a physiologically normal level. However, ranges of concentration are also possible and it is anticipated that concentrations ranging from about 0.01 ng/ml to about 5 ng/ml will also give an increase depending on the specific activity of the recombinant or native GM-CSF preparation. It will be understood however that it might be found that concentrations outside of this might also lead to a beneficial effect.

A base medium to which the hGM-CSF is added might be any one known to the person skilled in the art.

Preferably, however, the medium is an embryo specific medium. Current practice for culturing embryos has developed considerably since early efforts utilizing general tissue culture media. Generally the embryo specific media have come to reflect the physiological condition of the female reproductive tract.

There are differences in the milieu of embryos as they traverse from the oviduct and to the uterus and it has become practice to utilize sequentially changing media to reflect that difference. It is now recognized that the requirements of pre-compacted embryos (before the 8 cell stage) is different to those of post compacted embryos. An early sequential complex media for extended culture was G1/G2 (Barnes et al., 1995) which has been developed and modified and has led to increased pregnancy rates after blastocyst transfer (Jones et al., 1998; Gardner et al., 1998).

There are a number of differences, however, most prominent to culture formulation is the finding that pre compacted embryos tend to generate energy by oxidation of pyruvate, lactate and amino acids. The presence of glucose as an energy source has an adverse effect on early stage embryo development but is beneficial to post-compacted embryos. The level of glucose present in media of post-compacted embryos is notably lower than levels used in general tissue culture media practice. General tissue culture media tends to a have a level of about 5.56 mM whereas so called media nominally suitable for post compacted embryos generally does not exceed about 3.15 mM. These levels of glucose may also be used for culturing early stage (pre-compaction) embryos but it is generally accepted that media with no or minimal glucose is preferred for pre-compaction embryos.

Similarly pyruvate in media for pre-compacted embryos tends to be present in lower levels than when it is utlilised in general tissue culture media. Thus levels of about 0.1 to 0.5 mM pyruvate are generally utilised for media for pre-compacted embryos whereas when it is present in typical tissue culture media it is present at levels of 1 mM or greater. Typically pyruvate is absent in media for post-compacted embryos.

Lactate might also be used as an energy source for cultivation of embryos, and in particular for cultivation of pre-compacted embryos. Embryo specific media might have lactate present at suitable levels generally at levels of at least 0.1 mM and perhaps more typically at levels of about 10 mM or 12 mM but the levels might be as high as 30 mM.

Additionally it is found that the presence of a metal ion chelator such as EDTA or transferrin stimulates cell proliferation and compaction of early stage embryos. The benefits of EDTA in media for post-compacted embryos is more equivocal, consequently only media for pre-compacted embryos tends to have EDTA present. General tissue culture media do not have metal ion chelators present.

A more general difference in embryo specific media compared to general tissue culture media relates to the source of protein and/or complex growth factors. Early media contained serum such as horse or calf serum and, for compatibility, filtered heat inactivated maternal serum was used. This component is however of variable composition and quality and additionally has associated with its inclusion the potential for viral transmission. Present embryo specific media utilise purified albumin (Ashwood-Smith et al., 1989) recombinant serum albumin (Hooper, 2000) and glucosaminoglycan molecules (Gardner et al., 1999). Preferably recombinant serum albumin is used and is recombinant human serum albumin.

It has also become preferred to include stabilised derivatives of glutamine in embryo specific media. These prevent ammonia accumulation which otherwise impair the growth of or are toxic to embryos. Glutamine (1 mM) is routinely present in most tissue culture media but is labile. Examples of stabilised derivatives of glutamine include alanyl-glutamine and glycyl-glutamine. Alternatively if glutamine is used it is preferably present at levels of less than about 0.2 mM.

Preferred embryo specific media for pre-compaction embryos also have present thiol antioxidants. Preferably these antioxidants are selected from the group consisting of taurine or hypotaurine. Taurine has been described as having protective and embryogenic functions (Bavister et al., Preimplantation Embryo Development, Springer Verlag, NY, 1993; Dumoulin et al., Biol Reprod, 56: 739-744, 1997). Taurine acting as an osmolyte may be especially important when glucose is absent (Behr et al., 1999).

Certain forms of embryo specific media also includes hyaluronan in particular for S1 media (Scandinavian IVF Science AB, now known as Vitrolife AB, Gothenburg, Sweden;) and derivatives thereof, designed to support growth of cleavage stage embryos up to about the 8-cell stage. (S1 media is also referred to as IVF50). Hyaluronan is thought to promote embryo recovery of cryopreserved embryos after thawing, to enhance embryo development and blastocyst implantation. Additionally it is desirable that the media exhibits an absence of corticosteroids, for example hydroxycortisone, cortisone or dexamethosone because these are considered to be detrimental to embryo development. Additionally it is preferred that inorganic phosphates are absent or present in lower quantities, because their presence may reduce ATP synthesis in S1 or other pre-compaction stage media, and is particularly preferred when glucose is absent.

Another distinguishing feature of embryo culture media and somatic cell media is often the absence in embryo cell systems of purines and pyridines, which are sometimes, not always, present in formulations of somatic cell media.

Certain cytokines other than GM-CSF have also been shown to have an impact on embryo development and it might be desired to include these in the media of the present invention. These growth factors include, but are not limited to, leukaemia inhibitory factor (LIF) (Dungilson et al., 1996), epidermal growth factor (EGF) (Martin et al., 1998) and insulin like growth factor I (IGF-1) (Lighten et al., 1998).

A suitable medium might include but is not limited to HTF medium and modifications thereof (Quinn, 1985a), IVF50 (also known as S1, Scandinavian IVF Science), S2 (Scandinavian IVF Science), G1.2 (Scandinavian IVF Science) and G2.2 (Scandinavian IVF Science) which references are incorporated herein by references in relation to the media. Other commercially available media suitable for the present invention include Quinn's Advantage Series (Sage Biopharma, USA) Sydney IVF media (Cook Australia) Universal IVF, ISM+ series and BlastAssist media (Medi-Cult, Denmark) and HTF and P-1 (Irvine Scientific, USA).

In a broad form of a second aspect, the invention could be said to reside in a method of growing early stage human embryos to transfer ready blastocysts, including the step of incubating the embryos in vitro in a culture medium containing an effective amount or human GM-CSF for a time and under conditions to increase the proportion of transfer ready blastocysts.

It is anticipated that the embryos will generally be contacted with GM-CSF at an early stage. The early stage of the embryos may be from during or immediately after fertilisation, through to several days after fertilisation but preferably before 4 days. Most preferably the contact will be within 24 h of fertilisation. It will be understood that these embryos will be at the 1-16 cell, morula or pre-blastocyst stage of development. Generally an embryo cultured for 1 day will have 2 cells, an embryo cultured for 3 days will have 12-16 cells and will be compacted (morula stage), and an embryo cultured for 4 to 5 days will have developed to blastocyst stage. A blastocyst is characterised by a clearly visible blastoceol cavity.

It is anticipated that the in vitro growth will be continued until the blastocysts reach the day 4 to 6 stage, however, in certain embodiments of the invention the culturing of the embryo may be to an earlier, or later stage.

In one form this second aspect of the invention comprises culturing of the embryo in a serum deprived medium including human GM-CSF until about 8-cell or morula stage is reached, and then transferring to a second medium including human GM-CSF for further culturing to blastocyst stage.

In a broad form of a third aspect the invention could be said to reside in an IVF program comprising the steps of:

contacting an human egg with a human sperm to form a conceptus

growing the resulting conceptus at least after the 8 cell stage embryo has formed in vitro in a defined culture medium containing an effective amount of human GM-CSF for a time and under conditions to increase the chance of achieving a transfer ready blastocyst

transferring the transfer ready blastocyst into a compatible human uterus

For a better understanding, the invention will now be described with reference to a number of examples.

### BRIEF DESCRIPTION OF THE DRAWINGS

FIG. 1: Effect of GM-CSF on development of embryos to blastocysts, according to embryo grade. Data is the number of embryos developed to blastocyst from experiments 1, 2 and 3 combined, expressed as a percentage of the initial number of cleaved (2-4 cell) embryos. The number of embryos in each group are given in parentheses.

FIG. 2: The effect of GM-CSF on the development of embryos to blastocyst, hatching and attachment stages. Data is the number of embryos developed to or beyond each stage, from experiments 1, 2 and 3 combined, expressed as a percentage of the initial number of cleaved (2-4 cell) embryos. 2-C=2-cell embryos; 8-C=8-cell embryos; M=morulla; B=blastocyst; Exp B=expanded blastocyst; H=hatching; A=attached with trophectoderm outgrowth.

FIG. 3: The effect of GM-CSF on the rate of development of embryos to blastocyst. Data is the number of blastocysts at each time point, from experiments 1, 2 and 3 combined, expressed as a percentage of the total number of blastocysts at 144 h post insemination.

FIG. 4 RT-PCR analysis of GM-CSF receptor mRNA expression in human blastocysts. Total cellular RNA was extracted from TF-1 cells and each of two cohorts of blastocysts (Bφ1 and Bφ2), reverse transcribed by random priming and amplified by PCR with GM-Rα, GM-Rβ or actin-specific primers using conditions listed in Table 7.

FIG. 5 The effect of GM-CSF on the rate of development of embryos to the blastocyst stage; (A) an early blastocyst (day 5, 112 h post-insemination) from the control group; (B) an expanded blastocyst (day 5, 112 h post-insemination) cultured in rhGM-CSF; (C) a fully hatched blastocyst attached to the culture dish (day 6, 144 h post-insemination); (D) an attached blastocyst cultured in rhGM-CSF showing trophectoderm outgrowth (arrow; day 8, 200 h post-insemination).

FIG. 6 The effect of GM-CSF on the number of total cells (TCN), inner cell mass (ICM) cells and trophectoderm (TE) cells in day 5 blastocysts (120-124 h post-insemination). Values are mean±SD of blastocysts cultured in 2 ng/ml rhGM-CSF (n=11) and blastocysts cultured in media alone (n=10).

### DETAILED DESCRIPTION OF THE INVENTION

### EXAMPLE 1

Measurement of Embryonic Viability and Development

Materials and Methods

The embryos used in this study were donated by couples undergoing IVF treatment at Fertilitetscentrum AB, Göteborg, Sweden. Embryos frozen at the 2-4 cell stage were thawed at or beyond their one year storage limit in liquid nitrogen. Ethics approval for the study was obtained from the research ethics committee at University of Göteborg (number 700-96).

Embryo Media

The media use are essentially as described in Gardner et al., (1996).

MEM=minimal essential medium

EAA=essential amino acids

NEAA=non-essential amino acids

hSA=human serum albumin, purified native

S1 media (also known as IVF50) for use with embryos from fertilisation to approximately 8 cell stage:

| | (mM) | |
|---|---|---|
| NaCl | 123.97 | |
| KCl | 4.69 | |
| MgSO₄.7H₂0 | 0.2 | |
| KH₂PO₄ | 0.37 | |
| CaCl₂.2H₂O | 2.04 | |
| NaHCO₃ | 25.0 | |
| Glucose | 0.5 | |
| Na D,L-lactate | 10.5 | |
| Na pyruvate | 0.32 | |
| penicillin | 0.06 | g/L |
| streptomycin | 0.05 | g/L |
| hSA | 4 | mg/ml |
| MEM NEAA | 1 | ml/100 ml |

S2 for use on embryos having 8 or more cells up to blastocyst stage

| | : (mM) | |
|---|---|---|
| NaCl | 123.97 | |
| KCl | 4.69 | |
| MgSO₄.7H₂0 | 0.2 | |
| KH₂PO₄ | 0.37 | |
| CaCl₂.2H₂O | 2.04 | |
| NaHCO₃ | 25.0 | |
| Glucose | 3.15 | |
| Na D,L-lactate | 6.0 | |
| Na pyruvate | 0.10 | |
| penicillin | 0.06 | g/L |
| streptomycin | 0.05 | g/L |
| hSA | 4 | mg/ml |
| MEM NEAA | 1 | ml/100 ml |
| MEM EAA | 2 | ml/100 ml |
| MEM Vitamins | 1 | ml/100 ml |

MEM EAA (50× preparation) is as follows:

| | | |
|---|---|---|
| L arginine-HCl | 6.32 | g/L |
| L-Cysteine.2HCl | 1,564 | g/L |
| L-Histidine.HCl.H₂O | 2.1 | g/L |
| L-Isoleucine | 2.625 | g/L |
| L-Leucine | 2.62 | g/L |
| L-Lysine.HCl | 3.625 | g/L |
| L-Methionine | 0.755 | g/L |
| L-Phenylalanine | 1.65 | g/L |
| L-Threonine | 2.38 | g/L |
| L-Tryptophan | 0.51 | g/L |
| L-Tyrosine | 1.8 | g/L |
| L-Valine | 2.34 | g/L |

MEM NEAA (100× preparation) is as follows:

| | | |
|---|---|---|
| L-Alanine | 0.89 | g/L |
| L-Asparagine.H₂O | 1.5 | g/L |
| L-Aspartic Acid | 1.33 | g/L |
| L-Glutamic Acid | 1.47 | g/L |
| Glycine | 0.75 | g/L |
| L-Proline | 1.15 | g/L |
| L-Serine | 1.05 | g/L |

MEM vitamins (100× preparation) is as follows:

| | | |
|---|---|---|
| Choline Chloride | 0.1 | g/L |
| Folic acid | 0.1 | g/L |
| myo inositol | 0.2 | g/L |
| Niacinamide | 0.1 | g/L |
| D Pantothenic Acid.½Ca | 0.1 | g/L |
| Pyridoxine.HCl | 0.1 | g/L |
| Riboflavin | 0.01 | g/L |
| Thiamine.HCl | 0.1 | g/L |
| NaCl | 8.5 | g/L |

Ovarian Stimulation and In Vitro Fertilisation

Patients received 300 μg buserelin gonadotrophin-releasing hormone agonist (GnRHa; Suprecur; Hoechst, Frankfurt, Germany) three times daily intranasally, starting 1 week before expected menses and lasting for two weeks. Down-regulation was confirmed by a serum estradiol content of <0.2 nmol/l. Patients were then given recombinant follicle stimulating hormone (r-FSH; Gonal-F; Serono Laboratories, Aubonne, Switzerland; 150-225 IU/day sub-cutaneously). The starting dose was dependent on the patient age and/or previous response during ovarian stimulation (Wikland et al., 1994). The ovarian response was monitored by ultrasound and serum estradiol levels as previously described (Bergh et al., 1997). GnRHa and rFSH were administered until there was at least one follicle >18 mm in mean diameter and two others >16 mm. Finally, oocyte maturation was triggered by one sub-cutaneous injection of 10 000 IU of hCG (Profasi; Serono Laboratories).

Oocytes were retrieved 36-38 h after hCG administration, assessed morphologically and fertilised in vitro. The embryos were cultured in IVF-50 (also known as S1, Scandinavian IVF Science) and frozen on day 2 using a 3-step propanediol cryo-preservation kit (Freeze Kit 1, Scandinavian IVF Science) according to the manufacturers instructions.

Recombinant GM-CSF

Recombinant human (rh)GM-CSF was obtained from R&D Systems Europe Ltd, Oxon, UK. The biological activity of the recombinant cytokine preparations was measured in a bioassay employing a GM-CSF responsive cell line (human myeloid TF-1 cell line), essentially as described by (Kitamura et al. 1989). Duplicate serial 1:2 dilutions were incubated with 2000 TF-1 cells in 200 ul of RPMI-1640 (Gibco) supplemented with 10% fetal calf serum (FCS; Commonwealth Serum Laboratories, Australia), 5×10⁻⁵ M β-mercaptoethanol and antibiotics. After 2 days, cultures were pulsed with 1 uCi of ³H thymidine (Amersham, Arlington Heights, Ill.) for 6 hours, harvested onto glass fibre paper using a Titretech automated cell harvester and radioactivity measured in a liquid scintillation beta counter.

Embryo Thawing, Allocation and Culture

Frozen 2-4 cell embryos were thawed in four steps using a propanediol method for embryo thawing (Thaw Kit 1, Scandinavian IVF Science) following instructions given by the manufacturer. The viable embryos were classified and graded according to criteria listed in Table 1.

TABLE 1
Embryo classification criteria

| Embryo Grade | Morphology |
|---|---|
| A | Regular blastomeres without fragments |
| B | Regular or irregular blastomeres, up to 30% fragments |
| C | Regular or irregular blastomeres, more than 30% |
| | fragments |
| D | 50% of the blastomeres dead after thawing |

To avoid bias the embryos were randomly allocated, with regard to patient and embryo grade, into the different culture groups (Table 2). The embryos were cultured in groups of five embryos per drop. To avoid the toxic effects of ammonium, released due to metabolism and breakdown of amino acids, the culture media was renewed every 48 h until hatching occurred. In two experiments the embryos were cultured in 20 μl drops of IVF-50 (Scandinavian IVF Science) containing 2 ng/ml rhGM-CSF (diluted 1:25 000 from stock material) or carrier (2 ng/ml BSA, diluted 1:1 000 from stock material). Culture drops were covered by 4 ml Ovolil-200 (Scandinavian IVF Science) in Falcon 3004 dishes (Becton-Dickinson Labware, Franklin Lakes; N.J., USA). When blastocysts were detected these were transferred into 1 ml of S2 (Scandinavian IVF Science) in Falcon 3037 dishes, containing 5% FCS and 2 ng/ml rhGM-CSF or carrier. Developmental stage was scored every 8 h from thawing until 2300 h on day 8 (200 h post-insemination).

In a third experiment the embryos were transferred from IVF-50 into S2 medium (Scandinavian IVF Science) at the 6-8 cell stage. Additions of GM-CSF and carrier were the same as in the two previous experiments. When blastocysts were detected they were transferred to S2 medium with GM-CSF or carrier in Falcon 3037 dishes, coated 24 h previously with Biomatrix EHS (Boehringer Ingelheim Bioproducts, Heidelberg, Germany). Developmental stage was scored every 8 h from thawing until 2300 h on day 8 (200 h post-insemination). Embryo scoring in each of the experiments was performed by the same person (CS).

Statistical analysis was performed using Fisher's exact test and independent samples t-test (StatSoft, Inc.). Differences in data were considered significant when P<0.05.

TABLE 2
Distribution of grades amongst thawed 2-4 cell embryos

| Embryo grade | Control (%) | GM-CSF (%) |
|---|---|---|
| A | 20 | 20 |
| B | 22 | 27 |
| C | 32 | 33 |
| D | 26 | 20 |
| N | 50 | 49 |

Grades are defined in Table 1.

Results

The rate and extent of development of 2-4 cell embryos to the blastocyst and hatching blastocyst stages was significantly increased by the addition of rhGM-CSF to culture medium (Table 3).

TABLE 3
Rate and extent of embryo development in the
presence or absence of rhGM-CSF

| | | % BΦ | T₅₀ | | | % BΦ | T₅₀ | |
|---|---|---|---|---|---|---|---|---|
| Expt | n | Control | | % H | N | RhGM-CSF | | % H |
| 1 | 16 | 38 | 122 | 50 | 15 | 60 | 121 | 89 |
| 2 | 16 | 38 | 116 | 50 | 16 | 81 | 98 | 100 |
| 3 | 18 | 17 | 127 | 33 | 18 | 83 | 105 | 53 |
| Total | 50 | 31 | 122 | 47 | 49 | 76^{a} | 108^{b} | 78^{c} |

% BΦ = % of viable thawed 2-4 cells reaching blastocyst stage.
^{a}p < 0.0001
T₅₀ = number of hours post-insemination at which 50% blastocysts develop.
^{b}p = 0.0002 at 112 h PI
% H = % of blastocysts which fully or partially hatch.
^{c}p = 0.009

A comparison between the proportion of embryos reaching blastocyst stage and beyond in experiment 1 and 2 (culture media containing 5% FCS from day 5) and experiment 3 (serum-free media) are presented in Table 4. There are no significant differences between the two groups, showing that the beneficial effect of GM-CSF is not dependent on the presence of FCS.

TABLE 4
Percent embryos developing up to or beyond each
developmental stage in experiment 1 and 2 (FCS added)
compared with experiment 3 (no FCS added).

| | | | % | % | |
|---|---|---|---|---|---|
| | N | % BΦ | Exp BΦ | Hatching | Attached |
| Control (exp 1 + 2) | 32 | 37 | 28 | 19 | 0 |
| GM-CSF (exp 1 + 2) | 31 | 71 | 68 | 68 | 38 |
| Control (exp 3) | 18 | 17 | 6 | 6 | 0 |
| GM-CSF (exp 3) | 18 | 83 | 61 | 44 | 22 |

BΦ = blastocyst;
Exp BΦ = expanded blastocyst

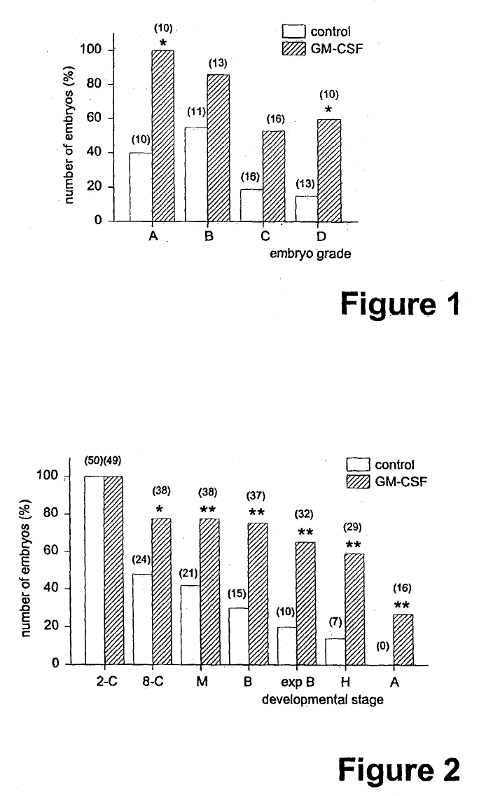

Although fewer poor quality embryos (grades C & D) reach blastocyst stage than good quality (grades A & B), GM-CSF exerted a comparable effect in all groups, with similar or slightly higher increases in the proportion of poor compared with good quality embryos achieving blastocyst stage (FIG. 1).

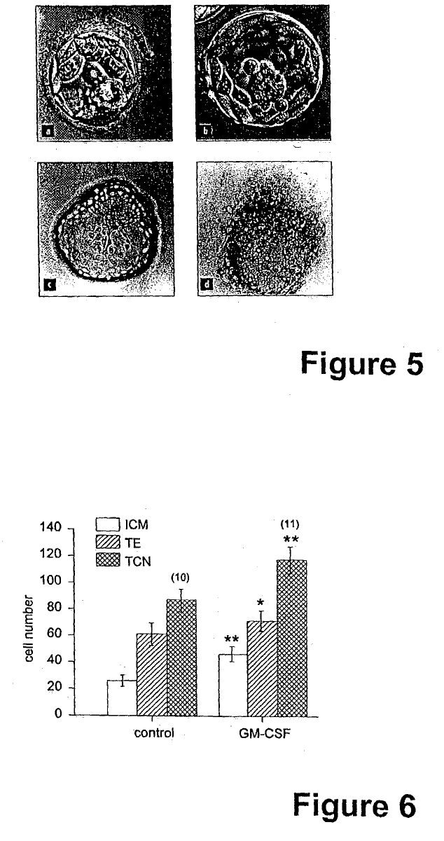

The majority of embryos grown in media alone were lost at the 4-16 cell stage. The beneficial effect of GM-CSF on blastocyst development appeared to result from rescue of this loss, with an 80% increase in the numbers of embryos reaching the morula stage of development (FIG. 2). Furthermore, the developmental potential of blastocysts was increased by culture in GM-CSF, since the rate of hatching was greater for embryos grown in GM-CSF. Similarly, blastocysts grown in GM-CSF (15/29), but not in control media (0/15), attached to the culture dish and showed trophoblast outgrowth (FIG. 2 and FIG. 5).

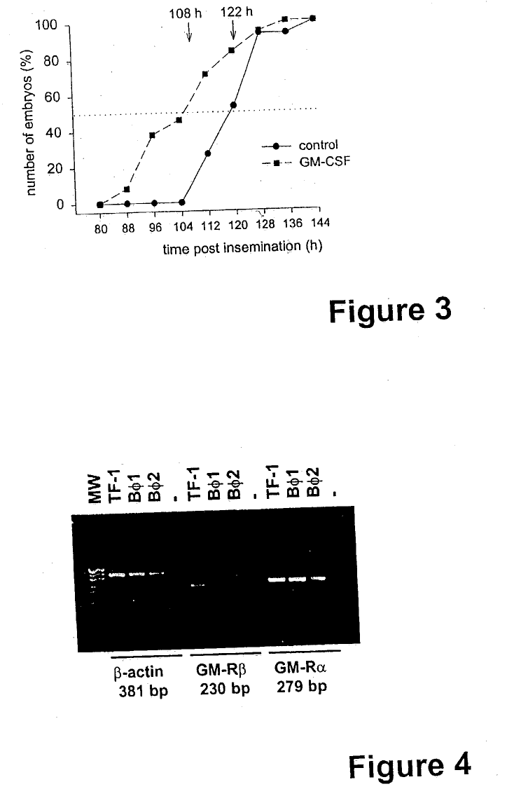

Finally, embryos cultured in the presence of rhGM-CSF had a significantly accelerated er rate of development, with 50% blastocyst development achieved 14 hours earlier in GM-CSF compared with the control group (FIG. 3).

Conclusions

These results support the hypothesis that GM-CSF secreted into the female reproductive tract during early pregnancy promotes embryo growth and development. The addition of GM-CSF to culture media promotes the formation of blastocysts even with poor post thaw quality embryos. Our results also show a beneficial effect of GM-CSF on blastocyst expansion, hatching, attachment and trophectoderm outgrowth. Although the functional significance of hatching in vitro is unknown, blastocyst expansion is one of the best criteria for blastocyst viability and developmental potential.

The cleavage rate of embryos is suggested to be an indicator of embryo quality (Shoukir et al., 1997), and the rate of embryo development is believed to be faster in vivo. Importantly, development of embryos to blastocysts was achieved significantly faster in the presence of rhGM-CSF.

### EXAMPLE 2

Measurement of Embryonic Viability and Development—Variation of Media and Source of GM-CSF

Materials and Methods

The embryos used in this study were donated by couples, after ovarian stimulation and in vitro fertilisation, as described in Example 1. For culture experiments, embryos frozen at the 2-4 cell stage were thawed at or beyond their one year storage limit in liquid nitrogen. The blastocysts used for the differential staining experiment were cultured from excess embryos, surplus to treatment and freezing requirements.

Recombinant GM-CSF

Two different commercial sources of recombinant human (rh)GM-CSF were used in these experiments. A laboratory grade preparation was obtained from R&D Systems Europe Ltd, Oxon, UK, and a pharmaceutical grade preparation, Molgramostim (Leucomax) was obtained from Schering & Plough, Madison, N.J., USA. The biological activity of both recombinant cytokine preparations were measured in a bioassay employing a GM-CSF responsive cell line (human myeloid TF-1 cell line), as described in Example 1.

Embryo Thawing, Allocation and Culture

Frozen 2-4 cell embryos were thawed and allocated randomly to experimental groups as described in Example 1. Embryo culture was performed as described in Example 1, in two different sequential media systems using two different commercial sources of rhGM-CSF. After thawing, the embryos were cultured first in G1.2 (Scandinavian IVF Science) or IVF-50. At 6-8 cell stage the embryos were transferred into G2.2 (Scandinavian IVF Science) or S2. The experiment included 6 groups: (a) G1.2/G2.2 alone, (b) G1.2/G2.2 containing 2 ng/ml rhGM-CSF (R&D Systems) (c) G1.2/G2.2 containing 2 ng/ml Molgramostim (Schering & Plough; diluted 1:75 000 from stock material), (d) IVF-50/S2 alone, (e) IVF-50/S2 containing 2 ng/ml rhGM-CSF (R&D Systems) (f) IVF-50/S2 containing 2 ng/ml Molgramostim. Developmental rate was scored every eighth hour until expanded blastocyst stage. Blastocysts were scored on day 5 at 120 h post-insemination according to criteria described previously (Dokras et al., 1993). Briefly, grade A blastocysts exhibited an expanded cavity with a distinct trophectoderm (TE) and an eccentrically located inner cell mass (ICM); grade B blastocysts were not yet expanded but otherwise morphologically identical to A; and grade C blastocysts exhibited poor morphology characterised by a number of degenerative foci in the ICM and TE and a poorly developed blastocoel cavity. Embryo scoring in each of the experiments was performed by the same person (CS).

Statistical analysis was performed using Fisher's exact test and independent samples t-test (StatSoft, Inc.). Differences in data were considered significant when P<0.05.

Differential Labelling of Blastocysts

Differential labelling was performed using a modification of a protocol described previously (Handyside and Hunter, 1984). Human blastocysts were cultured from excess embryos, surplus to treatment and freezing. On day 5 of culture (120-128 h post-insemination) the zona was removed in Acid Tyrodes solution containing 4 mg/ml PVP (360 000 Mw) and embryos were washed once in Gamete-100 (Scandinavian IVF Science) and three times in albumin-free S2 containing 4 mg/ml PVP (S2-PVP). The blastocysts were incubated in trinitro-benzene sulfonic acid (TNBS, Sigma Chemical Co., St Louis, Mo., USA; 10 mM in S2-PVP pH 8.5, 4° C./20 min in the dark) and washed three times in Gamete-100. TNBS-treated blastocysts were incubated in anti-dinitro-phenyl antibody (anti-DNP; Sigma, 0.2 mg/ml diluted in Gamete-100; 37° C./30 min) Embryos were then washed and incubated in guinea pig complement serum (Sigma; diluted 1:10 in Gamete-100; 37° C./30 min). Embryos were washed again and labelled with flourochromes (Sigma; 0.05 mM bisbenzimide and 10 ug/ml propidium iodide in Gamete-100, 37° C./30 min). After extensive washing embryos were fixed briefly in 1% paraformaldehyde and 0.5% glutaraldehyde in PBS, mounted under cover-slips in 20% glycerol in PBS and examined by fluorescence microscopy using a 400 nm exitation filter. Nuclei stained pink were scored as lysed trophectoderm cells (TE) and blue nuclei were scored as viable inner cell mass cells (ICM).

Results

This experiment demonstrates the effect of culture media and source of recombinant cytokine on GM-CSF stimulated blastocyst development. Cytokine formulations in two different sequential culture media systems were found to have equivalent bioactivities in the TF-1 cell proliferation assay (data not shown). There were no significant differences between the blastulation rates achieved in the two different culture media systems (Table 5). Both the rate and extent of development of 2-4 cell embryos to blastocysts was significantly increased by the addition of 2 ng/ml rh GM-CSF. The effect was comparable in extent in both G1.2/G2.2 and IVF-50/S2 sequential media combinations. Furthermore, the improvement in blastocyst development was achieved irrespective of the formulation of recombinant cytokine. The results also show that although culture in rhGM-CSF gives rise to more blastocysts, the distribution in morphological grade was comparable in treatment and control groups (Table 5).

TABLE 5
The effect of culture media and source of recombinant cytokine on
GM-CSF stimulated blastocyst development.

| | N | % Bφ | A/B/C (%) |
|---|---|---|---|
| G1.2/G2.2 alone | 23 | 30 | 57/29/14 |
| G1.2/G2.2 + rhGM-CSF (R&D) | 21 | 71** | 67/20/13 |
| G1.2/G2 + Molgramostim | 19 | 63* | 67/17/17 |
| IVF-50/S2 alone | 38 | 37 | 57/29/14 |
| IVF-50/S2 + rhGM-CSF (R&D) | 38 | 79*** | 67/26/7 |
| IVF-50/S2 + Molgramostim | 20 | 65* | 70/15/15 |

*P < 0.05;
**P < 0.01;
***P < 0.001

The Effect of Culture in GM-CSF on Blastomere Number and Allocation.

To investigate the effect of culture with GM-CSF on cell number and allocation to inner cell mass and trophectoderm cell lineage, blastocysts cultured with and without rhGM-CSF were analysed by immunosurgery and differential staining. Blastocysts cultured in the presence of rhGM-CSF had a significantly higher total cell number compared to blastocysts cultured in media alone (FIG. 6). An increase in the number of trophectoderm cells, and particularly in the number of inner cell mass cells, each contributed to the greater cell number in GM-CSF stimulated blastocysts.

### EXAMPLE 3

IVF Media

The techniques used for embryo culture in IVF procedures have not changed a great deal since the 1980s. These procedures are set out most particularly in Kerin et al (1983), Trouson et al (1980), Trouson et al (1982), and Quinn et al (1985) which references are incorporated herein by references in relation to those procedures.

The media in which this invention might be used can be any media suitable for use for the in vitro support of embryo development and growth. These media might include but are not limited to HTF medium and derivatives thereof (Quinn, 1985a), IVF50 (Scandinavian IVF Science), S2 (Scandinavian IVF Science), G1.2 (Scandinavian IVF Science) and G2.2 (Scandinavian IVF Science) which references are incorporated herein by references in relation to the media.

### EXAMPLE 4

Method of IVF Treatment

IVF procedures have not changed a great deal since the 1980s. The procedures for IVF treatment used in this invention are standard procedures that are set out most particularly in Kerin et al (1983), Trouson et al (1980), Trouson et al (1982), and Quinn et al (1985) which references are incorporated herein by references in relation to those procedures.

### EXAMPLE 5

The Expression of GM-CSF Receptors by Human Pre-Implantation Embryos In Vitro

Material and Methods

The embryos used in this study were donated by couples, after ovarian stimulation and in vitro fertilisation, as described in Example 1. Excess human 2-4 cell embryos surplus to patients' requirements were cultured in 20 ml droplets of IVF-50 overlayed with paraffin oil. On day 3 (72 h post insemination) the embryos were transferred to S2. Embryos were collected at blastocyst stage of development. The embryos were washed in PBS, snap frozen in liquid nitrogen and stored at −70° C. prior to RNA extraction.

Total cellular RNA was extracted from human GM-CSF responsive myeloid cells (TF-1 cell line), and from two cohorts each of twenty blastocysts using a method described by (Arcellana-Panlilio & Schultz,1993). Residual chromosomal DNA was removed by treatment with RNAse-free DNAse (Boehringer Mannheim) for 60 min at 37° C. First strand cDNA synthesis was achieved by reverse transcription (RT) of RNA primed with random hexamers using a Superscript RNase H-reverse transcriptase kit (Gibco) essentially according to the manufacturer's instructions. Detection of mRNA by RT-PCR was performed using primer pairs specific for the α-chain and β-chain of the GM-CSF receptor (GM-Rα and GM-Rβ), and β-actin (detailed in Table 7) and reagents supplied in a Taq DNA polymerase kit (Biotech International Ltd., Perth), essentially as described previously. The number of cycles and annealing temperature used for each primer pair are also given in Table 7. To increase the sensitivity of the GM-Rβ PCR, a nested primer design was employed, wherein cDNA was amplified by 30 cycles with GM-Rb ‘external’ primers followed by 25 cycles with GM-Rβ ‘internal’ primers. Each PCR product was analysed by electrophoresis through a 2% agarose gel containing EtBr, and visualised by trans-illumination with UV-light. Gels were photographed and the size of the PCR products was verified by comparison of their relative mobility to molecular weight markers.

TABLE 7
Primer sequences

| | Gene- | | Posi- | | |
|---|---|---|---|---|---|
| | bank | | tion | Cycle | |
| | acces- | | in | numb/ | |
| Tar- | sion | | se- | annealing | Product |
| get | number | Primer sequence | quence | temp | size |
| β- | M12481 | 5′ | 48-67 | 35/62° C. | 372 bp |
| actin | | tgtgatggtgggtatgggtc | | | |
| | | 3′ | 400- | | |
| | | tagatgggcacagtgtgggt | 419 | | |
| GM- | M64445 | 5′ | 162- | 40/60° C. | 279 bp |
| Rα | | catgcttctcctggtgacaa | 181 | | |
| | | 3′ | 421- | | |
| | | gtgactccttcatgcagaca | 440 | | |
| GM- | M59941/ | external: | 142- | 30/65° C. | 428 bp |
| Rβ | M38275 | | 161 | | |
| | | 5′ | 550- | | |
| | | ctacaccagccacatcacct | 569 | | |
| | | 3′ | 239- | 25/65° C. | 230 bp |
| | | agtcctgaagccgcttgtag | 258 | | |
| | | internal: | 449- | | |
| | | | 468 | | |
| | | 5′ | | | |
| | | gagccagtgtcctgtgacct | | | |
| | | 3′ | | | |
| | | tggtcctggtcggtgctgat | | | |

Results

The expression of GM-CSF receptor expression by in vitro generated blastocysts was examined by RT-PCR. Each of two preparations of blastocysts were found to express mRNA for the α-chain of the GM-CSF receptor, but mRNA for the β-chain was not detected, even when a highly sensitive nested PCR protocol was used (FIG. 4).

Conclusions

Expression of GM-CSF receptor α-chain mRNA was detected in each of two blastocyst cDNA preparations. These results indicate that human blastocysts have the molecular capacity to bind and respond to GM-CSF. The expression of the α-subunit in the absence of the β-chain may benefit blastocyst glucose transport and thus optimise the culture environment. Increased glucose uptake is likely to promote blastomere metabolic activity, and hence cell division, and may also prolong cell survival through the prevention of apoptosis.

### REFERENCES

Arcellana-Panlilio & Schultz, 1993. Methods Enzymol. 225; 303-28.

Armstrong & Chaouat, 1989. Biol Reprod 40; 466-474

Ashwood-Smith et al., 1989, J In Vitro Fertil and Embr Trans. 32 215-217

Barnes et al., 1995. Human Reproduction, 10:3243-3247

Behr et al., 1999, Human Reproduction, 14:454-457

de Moraes & Hansen, 1997, Biol Reprod. 57; 1060-1065

Ding et al., 1994 Proc Natl Acad Sci USA. 91(7); 2537-41

Drake & Head, 1994, J Reprod Immunol 26; 41-56

Dunglison et al., 1996, Hum Reprod. 11; 191 196

Edwards and Purdy, 1982, (eds) 1982 Human conception in vitro Academic Press, London

Gardner et al., 1996, Fertility Sterility 65:349-53

Gardner et al., 1998, Human Reproduction 13:3434-3440

Gardner et al., 1999, Human Reproduction 14:2575-2580

Giacomini et al., 1995. Hum-Reprod. 10; 3259-63

Hill et al., 1987. J Immunol 139; 2250-2254

Hooper, 2000, Biology of Reproduction 62:S249

Jokhi et al., 1994, 26; 147-164

Jones et al., 1998, Human Reproduction 13:169-177

Kerin et al, 1983, Clin Reprod. Fertil, 2; 129-142

Lea & Clark, 1993. Biol Reprod 48;930-953

Lenton et al., 1988, Ann NY Acad Sci, 541; 498-509

Lighten et al., 1998, Human Reproduction 13:3144-3150

Loke et al., 1992, J Reprod Immunol, 22; 33-45

Martin et al., 1998 Human Reproduction 13 :1645-1652

Ménézo et al., 1990, Biol Reprod, 40; 301-306

Ménézo et al., 1992, Hum Reprod, 7; 101-106

Oliveness et al., 1955, Hum Reprod, 9; 2367-2373

Plachot et al., 1955 In Aburumieh et al (eds) IXth World Congress on In Vitro Fertilisation and Assisted Reproduction Monduzzi Editore, Bologna p 37

Quinn et al 1985, In Annals of N.Y. Acad. Sci. 442; 195-204.

Quinn et al 1985a, Fertil. amd Steril. 44; 493-498

Robertson et al., 1991, pp 191-206 in Molecular and Cellular Immunobiology of the Maternal Fetal Interface, Wegmann et al eds Oxford University Press)

Robertson et al., 1992. Biol Reprod 46; 1069-79.

Robertson et al., 1996 Biol Reprod 54; 183-196.

Robertson et al., 1998 The effect of GM-CSF deficiency on early embryonic development in mice. Proceedings of the 29^{th} Annual Conference of the Australian Society for Reproductive Biology. Shoukir et al., 1997. Hum. Reprod. 7: 1531-1536

Robertson et al., 1999 Biol Reprod 60; 251-261.

Robertson et al., 2001 Biol Reprod 64; 206-215.

Sjöblom et al, 2002 Biol Reprod. 67; 1817-1823.

Tartakovsky & Ben-Yair, 1991. Dev Biol 146; 345-352

Trouson et al, 1980, Fertil. Steril. 34; 431-438

Trouson et al, 1982, J reprod. Fertil. 64; 285-294

Trounson et al 1982a) In: Edward and Prudy (eds) Human conception in vitro. Academic Press, London, p 201-205

Trounson 1984. in Invitro Fertilization and Embryo Transfer, Churchill Livingstone, (Trounson & Wood eds) pp 111-130

Weinberg et al., 1988, Fert Steril, 50; 993-5

Zhao & Chegini, 1994. J Clin Endocrinol Metab. 2; 662-5.

1. An embryo specific culture medium for in vitro propagation of preblastocyst human embryos, said medium comprising glucose at a level of between 0 to 3.15 mM, and no serum or serum fractions, said medium further comprising an effective amount of human GM-CSF to increase the chance of implantation of the embryos; the amount of GM-CSF being sufficient to increase the proportion of blastocysts formed from preblastocyst embryos when compared to embryos incubated in a medium lacking GM-CSF.

2. An embryo specific culture medium as in claim 1 further comprising lactate as an energy source.

3. An embryo specific culture medium as in claim 1 wherein lactate is present at a level of greater than 0.1 mM

4. An embryo specific culture medium as in either claim 1 or 2 further comprising pyruvate at a level of between 0 to 0.5 mM

5. An embryo specific culture medium as in either claim 1 or 4 further comprising any one or more of the group consisting of, a metal ion chelator, a stabilized glutamine derivative, a thiol antioxidant, hyaluronan and purified or recombinant human serum albumin.

6. The medium for propagation of preblastocyst embryos according to either claim 1 or 5 said medium comprising either an absence of inorganic phosphate or an absence of corticosteriods.

7. The medium for propagation of preblastocyst embryos according to either claim 1 or 5 said medium comprising an absence of purines and pyrimidines.

8. The medium for propagation of preblastocyst embryos according to either claim 1 or 5 said medium comprising one or more growth factors selected from group consisting of LIF, EGF and IGF-1.

9. The medium for propagation of preblastocyst embryos according to claim 1 wherein the human GM-CSF is in purified form.

10. The medium for propagation of preblastocyst embryos according to claim 9 wherein the human GM-CSF is purified from a non-animal and non-human source.

11. The medium for propagation of preblastocyst embryos according to claim 10 wherein the human GM-CSF is purified from a recombinant micro-organism.

12. The medium for propagation of preblastocyst embryos according to claim 1 wherein the level of human GM-CSF in the medium is between 0.01 ng/ml and 5 ng/ml.

13. The medium for propagation of preblastocyst embryos according to claim 12 wherein the level of human GM-CSF in the medium is 0.01 ng/ml.

14. The medium for propagation of preblastocyst embryos according to claim 12 wherein the level of human GM-CSF in the medium is 2 ng/ml.

15 The medium for propagation of preblastocyst embryos according to claim 1 to propagate embyros to the stage of day 3 or 4 development said media comprising pyruvate at levels of about 0.1 to 0.5 mM, and lactate at a level of greater than 0.1 mM.

16. An embryo specific culture medium for in vitro propagation of preblastocyst human embryos, said medium comprising glucose at a level of between 0 to 3.15 mM, said medium containing no components selected from the group consisting of serum and serum fractions, said medium further comprising an effective amount of human GM-CSF to increase the chance of implantation of the embryos; the level of GM-CSF being between 0.01 ng/ml and 5 ng/ml.

17. An embryo specific culture medium as in claim 16 further comprising lactate as an energy source.

18. An embryo specific culture medium as in claim 16 wherein lactate is present at a level of greater than 0.1 mM

19. An embryo specific culture medium as in either claim 16 or 17 further comprising pyruvate at a level of between 0 to 0.5 mM

20. An embryo specific culture medium as in claim 16 or 17 further comprising one or more of the group of components consisting of pyruvate at a level of between 0 to 0.05 mM a metal ion chelator, a stabilized glutamine derivative, a thiol antioxidant, hyaluronan and purified or recombinant human serum albumin, said medium not containing components selected from the group consisting of inorganic phosphate and corticosteriods.

21. An embryo specific culture medium as in either claim 16 or 17 further comprising one or more of the group of components consisting of pyruvate at a level of between 0 to 0.5 mM, EDTA, transferrin, a stabilized glutamine derivative, taurine, hypotaurine, hyaluronan and purified or recombinant human serum albumin, said medium not containing components selected from the group consisting of inorganic phosphate and corticosteriods.

22. The medium for propagation of preblastocyst embryos according to either claim 16 or 21 wherein the level of human GM-CSF in the medium is 2 ng/ml.

23 The medium for propagation of preblastocyst embryos according to claim 16 to propagate embyros to the stage of day 3 or 4 development said media comprising pyruvate at levels of about 0.1 to 0.5 mM, and lactate at a level of greater than 0.1 mM.
